(19) United States
(12) Patent Application Publication
(10) Pub. No.: US 2021/0186430 A1
(43) Pub. Date: Jun. 24, 2021

(54) HEART RATE CORRECTION METHOD AND SYSTEM AND COMPUTER READABLE MEDIUM

(71) Applicant: Wistron Corporation, New Taipei City (TW)

(72) Inventor: Chih-Hao Chiu, New Taipei City (TW)

(73) Assignee: Wistron Corporation, New Taipei City (TW)

(21) Appl. No.: 16/805,844

(22) Filed: Mar. 2, 2020

(30) Foreign Application Priority Data
Dec. 24, 2019 (TW) 108147308

(51) Int. Cl. A61B 5/00; A61B 5/024; G16H 50/30

(52) U.S. Cl. CPC A61B 5/721; A61B 2562/0219; G16H 50/30; A61B 5/02405

(57) ABSTRACT

A heart rate correction method and system and a computer readable medium are provided. A heartbeat measurement signal and an acceleration detection signal are collected during a sampling time range. A credibility of an instant heart rate is determined to be credible or incredible based on the acceleration detection signal. An average heart rate is calculated by taking out one or more instant heart rate of which the credibility is determined to be credible. Whether a difference between the average heart rate and a reference heart rate exceeds a variation range is determined. When it is determined that the difference between the average heart rate and the reference heart rate exceeds the variation range, a corrected heart rate is obtained based on a correction value and the reference heart rate, and the corrected heart rate is taken as an output heart rate corresponding to the sampling time range.

### CROSS-REFERENCE TO RELATED APPLICATION

This application claims the priority benefit of Taiwan application serial no. 108147308, filed on Dec. 24, 2019. The entirety of the above-mentioned patent application is hereby incorporated by reference herein and made a part of this specification.

### BACKGROUND

### Technical Field

The invention relates to a correction method and system, and particularly relates to a heart rate correction method and system and a computer readable medium for heart rate detection.

### Description of Related Art

In recent years, along with the public's attention to health, a variety of heart rate monitoring devices has appeared on the market. In terms of existing technology, these devices are combined with an accelerometer to determine user's movements and calculate heartbeats by using different algorithms based on a static state or a moving state of the user.

For example, when the user is in the static state or a near static state (with a small motion) and an intensity of an acceleration detection signal is low, a heartbeat measurement signal is stable, and a static algorithm is generally used to take a regularity of the heartbeat measurement signal as the heart rate. Moreover, in order to prevent an unexpected noise from affecting a heart rate value, an average heart rate is output by taking several consecutive instant heart rates, and deleting the maximum and minimum values.

On the other hand, for example, when the user is in the moving state or a dynamic state (with a large motion), and the intensity of the acceleration detection signal is high, the heartbeat measurement signal is unstable and mixed with many noises of motions, at this moment, the heartbeat measurement signal does not only include heart rate information, so that a dynamic algorithm is generally applied. In the dynamic algorithm, the Fast Fourier Transformation (FFT) may be used to separate the signal, and then the most likely frequency is taken from the signal to calculate the heart rate. However, the above method may reduce a resolution of the heart rate. Moreover, another dynamic algorithm is to use a principle of subtracting the heartbeat measurement signal and the acceleration detection signal to output the heart rate, but such method is more suitable for a dynamic heart rate calculation method designed for regular motions (such as jogging).

However, through testing, when a heart rate measurement device is stationary, a heartbeat value of the heartbeat measurement signal is more reliable. While maintaining a regular motion (such as jogging), the heartbeat value of the heartbeat measurement signal is also relatively accurate. However, the heartbeat value of an irregular motion may be inaccurate. For example, during a process from standing to jogging of the user (a process from the static state to the dynamic state), the user's heartbeat values after initial standing and starting jogging are all accurate, but an accuracy of the heartbeat value obtained from the static state to a preparation motion the dynamic state in the middle is decreased. Further, irregular motions or short-term large motions in the static state may also cause inaccurate heartbeat value. For example, when the user sits in a chair at rest, the heartbeat value is accurate, and when the user suddenly turns the head, the accuracy of the heartbeat value drops. The reason is that the user has a motion, but the heart rate is still calculated and output by using the static algorithm as the acceleration detection signal has not reached a predetermined threshold.

Based on the above description, to use the static algorithm or the dynamic algorithm to calculate the heart rate for irregular motions in the static state may all reduce the accuracy of the heart rate. Therefore, there is no suitable algorithm for the irregular motion in the static state at present, which is the main reason for the heart rate inaccuracy in the static state.

### SUMMARY

The invention is directed to a heart rate correction method and system and a computer readable medium, which are adapted to output a relatively accurate heart rate even if there are irregular small motions or short-term large motions in a static state.

One embodiment of the invention provides a heart rate correction method including: collecting a heartbeat measurement signal during a sampling time range to calculate a plurality of instant heart rates corresponding to a plurality of sampling intervals included in the sampling time range; obtaining an acceleration detection signal during the sampling time range; determining whether a credibility of each of the instant heart rates is credible or incredible based on the acceleration detection signal; calculating an average heart rate by taking out the instant heart rates of which the credibility is determined to be credible; determining whether a difference between the average heart rate and a reference heart rate exceeds a variation range; and obtaining a corrected heart rate based on a correction value and the reference heart rate when it is determined that the difference between the average heart rate and the reference heart rate exceeds the variation range, and taking the corrected heart rate as an output heart rate corresponding to the sampling time range.

One embodiment of the invention provides a heart rate correction system including a heart rate sensor, an acceleration sensor and a processor. The processor is electrically coupled to the heart rate sensor, the acceleration sensor and a storage device, and the processor is configured to: collect a heartbeat measurement signal through the heart rate sensor during a sampling time range to calculate a plurality of instant heart rates corresponding to a plurality of sampling intervals included in the sampling time range; obtain an acceleration detection signal through the acceleration sensor during the sampling time range; determine whether a credibility of each of the instant heart rates is credible or incredible based on the acceleration detection signal; calculate an average heart rate by taking out the instant heart rates corresponding to the sampling intervals of which the credibility is determined to be credible; determine whether a difference between the average heart rate and a reference heart rate exceeds a variation range; obtain a corrected heart rate based on a correction value and the reference heart rate when it is determined that the difference between the average heart rate and the reference heart rate exceeds the variation range, and take the corrected heart rate as an output heart rate corresponding to the sampling time range.

One embodiment of the invention provides a heart rate correction system including a processor electrically coupled to a heart rate sensor and an acceleration sensor. The processor is configured to: collect a heartbeat measurement signal through the heart rate sensor during a sampling time range to calculate a plurality of instant heart rates corresponding to a plurality of sampling intervals included in the sampling time range; obtain an acceleration detection signal through the acceleration sensor during the sampling time range; determine whether a credibility of each of the instant heart rates is credible or incredible based on the acceleration detection signal; calculate an average heart rate by taking out the instant heart rates corresponding to the sampling intervals of which the credibility is determined to be credible; determine whether a difference between the average heart rate and a reference heart rate exceeds a variation range; obtain a corrected heart rate based on a correction value and the reference heart rate when it is determined that the difference between the average heart rate and the reference heart rate exceeds the variation range, and use the corrected heart rate as an output heart rate corresponding to the sampling time range.

One embodiment of the invention provides a computer readable medium storing a plurality of code snippets, and an electronic device loads the code snippets to execute following steps: collecting a heartbeat measurement signal during a sampling time range to calculate a plurality of instant heart rates corresponding to a plurality of sampling intervals included in the sampling time range; obtaining an acceleration detection signal during the sampling time range; determining whether a credibility of each of the instant heart rates is credible or incredible based on the acceleration detection signal; calculating an average heart rate by taking out the instant heart rates of which the credibility is determined to be credible; determining whether a difference between the average heart rate and a reference heart rate exceeds a variation range; obtaining a corrected heart rate based on a correction value and the reference heart rate when it is determined that the difference between the average heart rate and the reference heart rate exceeds the variation range, and taking the corrected heart rate as an output heart rate corresponding to the sampling time range.

Based on the above description, according to the embodiments of the invention, the output heart rate is adjusted based on the acceleration detection signal, and when the intensity of the acceleration detection signal is high, it means that the user has a large or short-term motion, at this moment, the previous heart rate value is used as a basis to adjust the heart rate to be output with reference of the current acceleration detection signal. In this way, the stable heart rate may be output even when there is a motion under the static state.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a block diagram of a heart rate correction system according to an embodiment of the invention.

FIG. 2 is a flowchart illustrating a heart rate correction method according to an embodiment of the invention.

FIG. 3 is a schematic diagram of corresponding curves of a heartbeat measurement signal and an acceleration detection signal in a sampling time range according to an embodiment of the invention.

FIG. 4 is a flowchart illustrating a heart rate correction method according to another embodiment of the invention.

FIG. 5 is a diagram illustrating curves of output heart rates before and after correction according to an embodiment of the invention.

### DESCRIPTION OF THE EMBODIMENTS

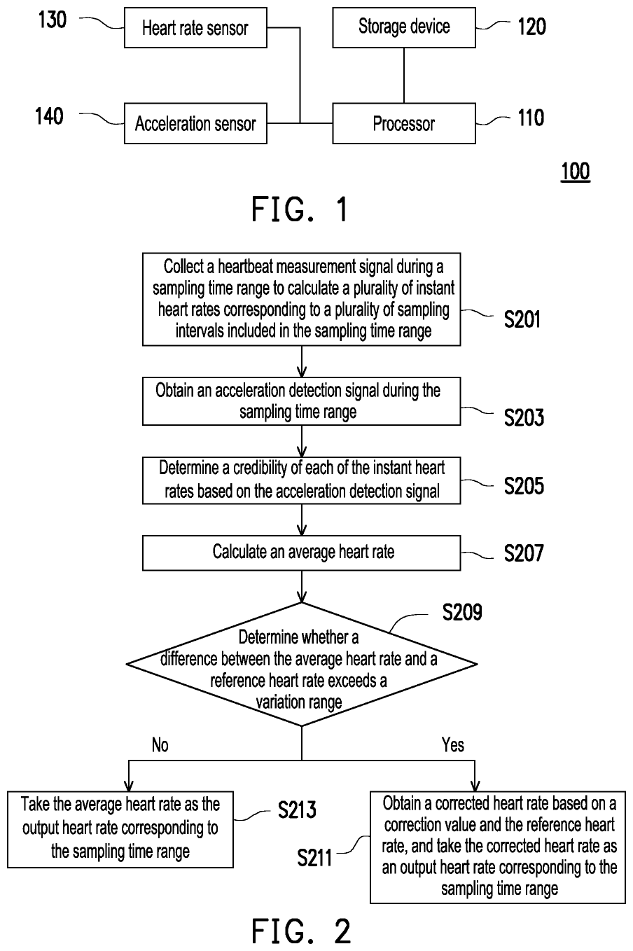

FIG. 1 is a block diagram of a heart rate correction system according to an embodiment of the invention. Referring to FIG. 1, the heart rate correction system 100 includes a processor 110, a storage device 120, a heart rate sensor 130 and an acceleration sensor 140. The processor 110 is electrically coupled to the storage device 120, the heart rate sensor 130 and the acceleration sensor 140, directly or indirectly.

The processor 110 is, for example, a Central Processing Unit (CPU), a Physics Processing Unit (PPU), a programmable microprocessor, an embedded control chip, a Digital Signal Processor (DSP), an Application Specific Integrated Circuits (ASIC) or other similar device.

The storage device 120 is, for example, any type of a fixed or movable Random Access Memory (RAM), a Read-Only Memory (ROM), a flash memory, a hard disk or other similar devices or a combination of the above devices. The storage device 120 stores a plurality of code snippets, and after the code snippets are loaded, the processor 110 executes the code snippets to implement a following heart rate correction method.

In an embodiment, the heart rate sensor 130 and the acceleration sensor 140 may be arranged in a same wearable device, and the storage device 120 and the processor 110 are disposed in an electronic device with a computation function such as a smart phone, a tablet PC, etc. The wearable device is used to detect heartbeats and acceleration values, and transmit an obtained heartbeat measurement signal and acceleration sensing signal to the electronic device, and the electronic device performs heart rate correction. The wearable device is, for example, a headset, a smart bracelet, a smart watch, etc. In other embodiments, the heart rate sensor 130 and the acceleration sensor 140 may also be arranged in different wearable devices/electronic devices, for example, the heart rate sensor 130 is disposed on a wearable device, and the acceleration sensor 140 is disposed on another different wearable device or disposed on the same electronic device as the processor 110.

Moreover, in other embodiments, the processor 110, the storage device 120, the heart rate sensor 130 and the acceleration sensor 140 may also be disposed in a same electronic device.

The heart rate sensor 130 is used for measuring heartbeats to obtain the heartbeat measurement signal. The heart rate sensor 130 is, for example, a sensor using photoplethysmography (PPG), but the invention is not limited thereto, and the heart rate sensor 130 may also be a radar sensor or an electrocardiogram (ECG) sensor. The acceleration sensor 140 is used for acceleration detection to obtain the acceleration sensing signal.

The heart rate correction system 100 mainly refers to the acceleration sensing signal to determine a credibility of an instant heart rate (iHR) at the moment of the sampling interval, and then corrects an output heart rate according to the credibility. An embodiment is provided below to describe various steps of a heart rate correction method with reference of the aforementioned heart rate correction system 100.

FIG. 2 is a flowchart illustrating a heart rate correction method according to an embodiment of the invention. In the embodiment, the processor 110 is configured to execute the code snippets stored in the storage device 120 to implement the following heart rate correction method.

Referring to FIG. 1 and FIG. 2, in a step S201, the processor 110 collects a heartbeat measurement signal through the heart rate sensor 130 during a sampling time range to calculate a plurality of instant heart rates corresponding to a plurality of sampling intervals included in the sampling time range. Moreover, in a step S203, the processor 110 obtains an acceleration detection signal through the acceleration sensor 140 during the sampling time range. The step S201 and the step S203 are simultaneously executed, i.e., the processor 110 simultaneously collects the corresponding data through the heart rate sensor 130 and the acceleration sensor 140. For example, the processor 110 collects the heartbeat measurement signal and the acceleration detection signal every 20 ms. The processor 110 may first calculate the instant heart rate of each sampling interval in the heartbeat measurement signal according to a space between two peaks forming each of the sampling intervals, and calculate an accumulated amount of acceleration values in each sampling interval in the acceleration detection signal.

Then, in a step S205, the processor 110 determines a credibility of each of the sampling intervals based on the acceleration detection signal. The processor 110 averages an accumulated amount of a plurality of acceleration signal values detected in each of the sampling intervals to obtain an acceleration average. Moreover, the processor 110 determines whether the credibility of each of the sampling intervals is credible or incredible based on the acceleration average of each of the sampling intervals. To be specific, if the acceleration average is too high, it represents that a large motion occurs in this sampling interval, and the heartbeat measurement signal in this sampling interval is incredible. In this way, the processor 110 compares the acceleration average with a credibility threshold. If the acceleration average is smaller than or equal to the credibility threshold, the credibility is determined to be credible. If the acceleration average is greater than the credibility threshold, the credibility is determined to be incredible. For example, a mark of “false” is given to the instant heart rate where the acceleration average is greater than the credibility threshold, otherwise, a mark of “true” is given.

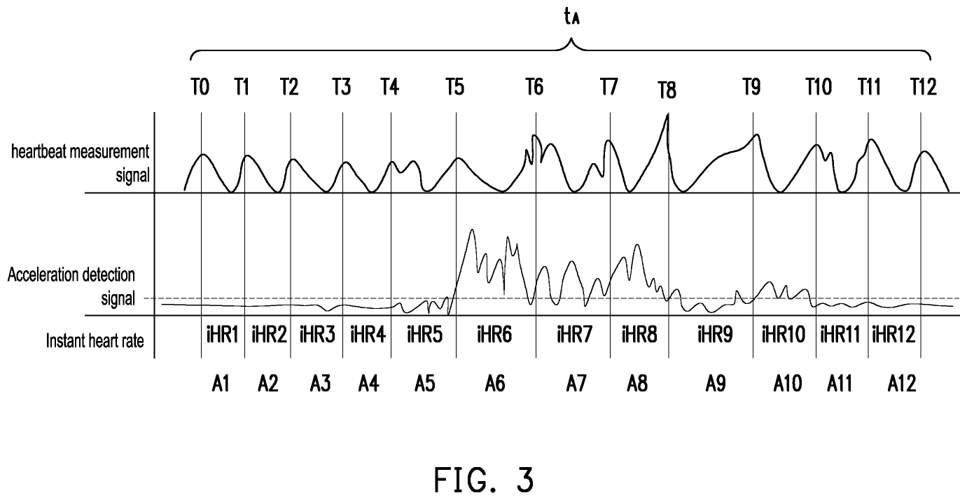

FIG. 3 is a schematic diagram of corresponding curves of the heartbeat measurement signal and the acceleration detection signal in a sampling time range according to an embodiment of the invention. Referring to FIG. 3, a sampling time range t_{A} (time axes T0-T12) includes 12 sampling intervals A1-A12.

The processor 110 calculates the respective instant heart rates iHR1-iHR12 corresponding to the sampling intervals A1-A12, respectively. The heart rate information may be obtained from photoplethysmography through two different analysis methods of time domain analysis and frequency domain analysis. For example, the heart rate information may be obtained by calculating a time from a previous peak to a next peak.

Moreover, the processor 110 averages an accumulated amount of a plurality of acceleration signal values respectively included in the sampling intervals A1-A12 to obtain the acceleration average of each of the sampling intervals A1-A12, and compares each of the acceleration averages with a credibility threshold TH. Then, the credibility of the instant heart rate corresponding to the acceleration average smaller than the credibility threshold TH is determined to be credible. The credibility of the instant heart rate corresponding to the acceleration average greater than the credibility threshold TH is determined to be incredible. For example, in FIG. 3, the credibility of the instant heart rates iHR1-iHR5, iHR9, iHR11, iHR12 of the sampling intervals A1-A5, A9, A11, A12 are determined to be credible, and the processor 110 gives each of the above instant heart rates a mark of “true”, and the credibility of the instant heart rates iHR6-iHR8, iHR10 of the sampling intervals A6-A8, A10 are determined to be incredible, and the processor 110 gives each of the above instant heart rates a mark of “false”.

Referring back to FIG. 2, in a step S207, the processor 110 calculates an average heart rate. The processor 110 calculates the average heart rate by taking out all of the instant heart rates of which the credibility is determined to be credible within the sampling time range. For example, referring to FIG. 3, the instant heart rates iHR1-iHR5, iHR9, iHR11, iHR12 corresponding to the sampling intervals A1-A5, A9, A11, A12 are taken out to calculate the average heart rate. Namely, the average heart rate of the sampling time range t_{A} shown in FIG. 3=(iHR1+iHR2+iHR3+iHR4+iHR5+iHR9+iHR11+iHR12)/8.

Then, in a step S209, the processor 110 determines whether a difference between the average heart rate and a reference heart rate exceeds a variation range. After obtaining the average heart rate, the processor 110 corrects the average heart rate according to the reference heart rate and the variation range determined in advance. For example, before the heart rate correction method is executed, the variation range of the heart rate, i.e. an acceptable range of the instant heart rate variation, is first determined. It is assumed that the determined variation range is 5, it represents that the output heart rate at this moment is expected to be within a range of the reference heart rate ±5. If the heart rate at this moment is N, the maximum heart rate at a next moment is N+5 and the minimum heart rate is N−5. The reason is that in terms of a physiological response of human body, the change in the heart rate is slow-rising or slowly-falling rather than an instant and drastic change, so that a heart rate variation amount within a short time is predictable.

When it is determined that the difference between the average heart rate and the reference heart rate exceeds the variation range, in a step S211, the processor 110 obtains a corrected heart rate based on a correction value and the reference heart rate, and takes the corrected heart rate as an output heart rate corresponding to the sampling time range. To be specific, the processor 110 calculates a sampling credibility corresponding to the sampling time range based on a number of the instant heart rates of which the credibility is determined to be credible, and then obtains the corresponding correction value from a correction table based on the sampling credibility.

In the embodiment, the processor 110 calculates the sampling credibility corresponding to the sampling time range based on the number of the instant heart rates of which the credibility is determined to be credible within the sampling time range. The sampling credibility is obtained based on a following equation:

Tr=N_true/N_sum.

Where Tr represents the sampling credibility, N_true represents the number of the instant heart rates of which the credibility is determined to be credible within the sampling time range, and N_sum represents a total number of all of the instant heart rates included in the sampling time range.

The corrected heart rate is obtained based on a following equation:

The corrected heart rate=the reference heart rate±the correction value.

The correction value is determined to be positive or negative according to the average heart rate and the reference heart rate. If the average heart rate is greater than the reference heart rate, the correction value is positive, and the corrected heart rate=the reference heart rate+the correction value. Conversely, if the average heart rate is smaller than the reference heart rate, the correction value is negative, and the corrected heart rate=the reference heart rate—the correction value.

Referring to a table 1, the table 1 is one of implementations of the correction table. The correction table is established in advance in the heart rate correction system 100. The correction table records a plurality of credibility ranges and their corresponding correction values, and the correction values are set according to the variation ranges.

TABLE 1

| Credibility range | Correction value |
|---|---|
| 80%~100% | 5 |
| 60%~80% | 4 |
| 40%~60% | 3 |
| 20%~40% | 2 |
| 0%~20% | 1 |

A correction logic of the table 1 may be regarded as: in the case of a high sampling credibility, although the heart rate variation exceeds an expectation, the trend of large heart rate variation is credible, so that a higher correction value is given; in the case of a low sampling credibility, the trend of large heart rate variation is incredible, so that a lower correction value is given.

Regarding the FIG. 3, the sampling time range t_{A} includes 12 instant heart rates (iHR1-iHR12) in total, among which the credibility of 8 instant heart rates (iHR1-iHR5, iHR9, iHR11, iHR12) are determined to be credible. Therefore, N_true is 8, and N_sum is 12. The sampling credibility Tr of the sampling time range t_{A} is 8/12=67%. The sampling credibility Tr of the sampling time range t_{A} falls within the credibility range of 60%-80%, so that when it is determined that the difference between the average heart rate and the reference heart rate exceeds the variation range, in the step S211, the processor 110 takes out the corresponding correction value 4 according to the sampling credibility Tr to perform correction.

On the other hand, when it is determined that the difference between the average heart rate and the reference heart rate does not exceed the variation range, i.e., when the difference falls within the variation range, as shown in a step S213, the processor 110 directly takes the average heart rate as the output heart rate corresponding to the sampling time range.

In the embodiment, the reference heart rate is set to a predetermined value in advance. However, in other embodiments, the currently or previously obtained output heart rate may also be used as a next reference heart rate. Another example is provided below for further description.

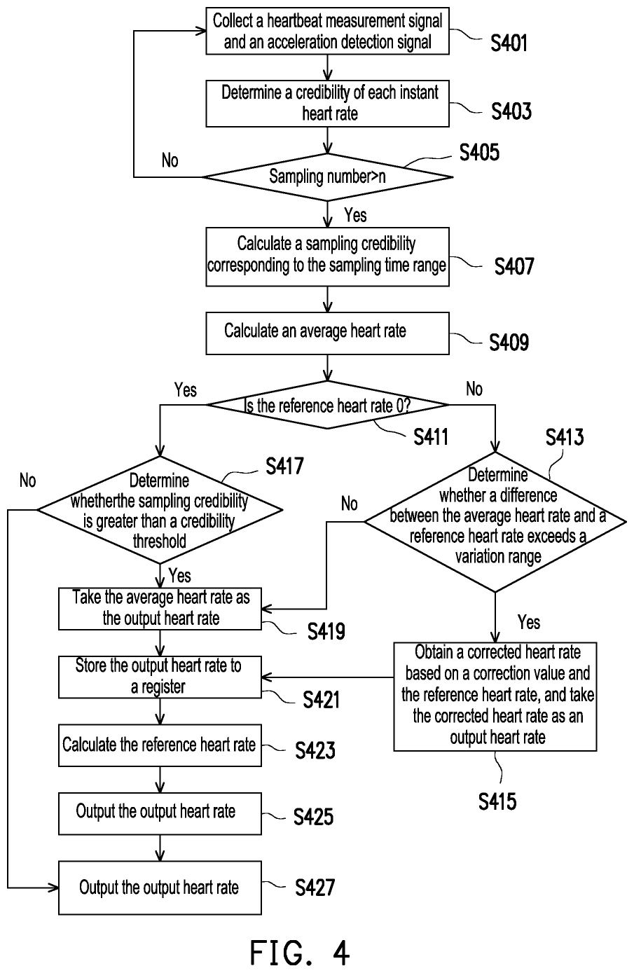

FIG. 4 is a flowchart illustrating a heart rate correction method according to another embodiment of the invention. Referring to FIG. 1 and FIG. 4, in a step S401, the processor 110 respectively collects a heartbeat measurement signal and an acceleration detection signal through the heart rate sensor 130 and the acceleration sensor 140 within the sampling time range. The processor 110 calculates the instant heart rate iHR in the acceleration detection signal according to a distance between two peaks forming a sampling interval, and counts an accumulated amount of acceleration values in the sampling interval in the acceleration detection signal.

Then, in a step S403, the processor 110 determines a credibility of the instant heart rate. The step S403 is similar to the step S205, and the processor 110 averages accumulated amounts obtained by accumulating of a plurality of acceleration signal values detected in the sampling interval to obtain an acceleration average. Then, the acceleration average is compared with a credibility threshold. If the acceleration average is smaller than or equal to the credibility threshold, the credibility is determined to be credible. If the acceleration average is greater than the credibility threshold, the credibility is determined to be incredible.

Then, in a step S405, the processor 110 determines whether a sampling number is greater than n. Namely, the processor 110 accumulates the sampling number by 1 each time when completing determination of the instant heart rate of one sampling interval. If the sampling number is not greater than n, the processor 110 continually takes out a next sampling interval to determine the credibility of the instant heart rate thereof, i.e., to repeat the step S401 and the step S403. If the sampling number is greater than n, in a step S407, the processor 110 calculates the sampling credibility corresponding to the sampling time range (n sampling intervals).

In the step S405, a more stable result may be obtained by counting multiple instant heart rates over a period of time, which may reduce an affect on a single instant heart rate fluctuation caused by motion. As shown in FIG. 3, taking n=12 as an example, i.e. the sampling number is 12 instant heart rates, i.e. no output heart rate is generated in 11 sampling intervals (A1-A11) at an initial startup, and the output heart rate is obtained at the 12^{th} sampling interval A12.

In the step S407, the processor 110 calculates the sampling credibility corresponding to the sampling time range based on the number of the instant heart rates of which the credibility is determined to be credible within the sampling time range, where the sampling credibility is obtained according to a following equation:

Tr=N_true/N_sum.

Where, Tr represents the sampling credibility, N_true represents the number of the instant heart rates of which the credibility is determined to be credible within the sampling time range, and N_sum represents a total number of all of the instant heart rates included in the sampling time range.

Regarding the FIG. 3, the sampling time range t_{A} includes 12 instant heart rates (iHR1-iHR12) in total, among which the credibility of 8 instant heart rates (iHR1-iHR5, iHR9, iHR11, iHR12) are determined to be credible. Therefore, the sampling credibility Tr of the sampling time range t_{A} is 8/12=67%.

Then, in a step S409, the processor 110 calculates an average heart rate. The step S409 is the same to the step S207, in the sampled instant heart rates, the processor 110 takes out all of the instant heart rates of which the credibility is determined to be credible within the sampling time range t_{A} to calculate an average value thereof to obtain the average heart rate. Referring to FIG. 3, the average heart rate of the sampling time range t_{A}=(iHR1+iHR2+iHR3+iHR4+iHR5+iHR9+iHR11+iHR12)/8.

Then, in a step S411, the processor 110 determines whether the reference heart rate is 0 or has no value. Here, the reference heart rate is obtained based on a plurality of output heart rates. To be specific, a temporary storage is configured in the heart rate correction system 100, and the temporary storage is used to store the obtained multiple output heart rates. The determination of whether the reference heart rate is 0 or has no value may be achieved by determining whether any output heart rate is stored in the temporary storage. Namely, in case that no output heart rate is stored in the temporary storage, the reference heart rate is 0 or has no value. Conversely, if any output heart rate has been stored in the temporary storage, the reference heart rate is not 0 or has a value.

Further, when it is determined that no output heart rate is stored in the temporary storage, the processor 110 does not correct the average heart rate but directly takes the average heart rate as the output heart rate for storing into the temporary storage. Moreover, when it is determined that any output heart rate is stored in the temporary storage, the processor 110 calculates a heart rate average of all output heart rates included in the temporary storage, i.e., accumulates all of the output heart rates included in the temporary storage for averaging, and takes the heart rate average as the reference heart rate to read the reference heart rate from the temporary storage for heart rate correction

If the reference heart rate is not 0, in a step S413, it is determined whether a difference between the average heart rate and the reference heart rate exceeds the variation range. If it is determined that the difference between the average heart rate and the reference heart rate exceeds the variation range, as shown in a step S415, the processor 110 obtains a corrected heart rate based on the correction value and the reference heart rate. On the other hand, when it is determined that the difference between the average heart rate and the reference heart rate does not exceed the variation range, i.e., the difference falls within the variation range, as shown in a step S419, the processor 110 directly takes the average heart rate as the output heart rate corresponding to the sampling time range.

The step S209, the step S211, and the step S213 of FIG. 2 may be respectively referred for detailed descriptions of the steps S413, S415, and S419.

Referring back to the step S411, if the reference heart rate is 0, it represents that the heart rate correction system 100 has not output any output heart rate, and in a step S417, the processor 110 determines whether the sampling credibility is greater than a credibility threshold. When it is determined that the sampling credibility is not greater than the credibility threshold, the average heart rate is not stored in the temporary storage, in a step S427, a heart rate correction of a next sampling time range is performed. When it is determined that the sampling credibility is greater than the credibility threshold, in the step S419 and a step S421, the average heart rate is directly taken as the output heart rate, and the output heart rate is stored in the temporary storage.

The step S417 is used to determine whether the average heart rate of the sampling time range (for example, the sampling time range t_{A} shown in FIG. 3) is credible. In case of incredible, the average heart rate is discarded (the average heart rate is not stored in the temporary storage), and in case of credible, the average heart rate is kept (the average heart rate is stored in the temporary storage). In the embodiment, the credibility threshold is set to 90%, and the average heart rate is kept when human body is almost in a static state.

After adding a new batch of output heart rate to the temporary storage, as shown in a step S423, the processor 110 recalculates the reference heart rate. The processor 110 may re-accumulate all of the output heart rates included in the temporary storage for averaging, and take the heart rate average as the reference heart rate.

Then, in a step S425, the processor 110 outputs an output heart rate. For example, the output heart rate is output to a display in a visual presentation manner, or the output heart rate is output to a speaker in an aural presentation manner.

Then, in a step S427, the processor 110 performs heart rate correction of a next sampling time range.

A following table 2 lists correction results of sampling time ranges t_{A}-t_{G} obtained by repeating the steps S401-S427. As shown in the table 2, the correction value corresponding to each of the sampling time ranges may be different according to the sampling credibility. Regarding the sampling time range t_{A}, the sampling credibility is within a credibility range of 60%-80%, so that the correspondingly used correction value is ±4. If the average heart rate is greater than the reference heart rate, the corrected heart rate=the reference heart rate+4. Conversely, if the average heart rate is smaller than the reference heart rate, the corrected heart rate=the reference heart rate-4, and the others are deduced by analogy.

TABLE 2

| | Sampling time range | | | | | | |
|---|---|---|---|---|---|---|---|
| | t_{A} | t_{B} | t_{C} | t_{D} | t_{E} | t_{F} | t_{G} |
| Time axes | T0~T12 | T13~T25 | T26~T38 | T39~T41 | T42~T54 | T55~T67 | T68~T80 |
| Output heart rate | HR1 | HR2 | HR3 | HR4 | HR5 | HR6 | HR7 |
| Sampling credibility | 8/12 | 6/12 | 2/12 | 8/12 | 6/12 | 8/12 | 11/12 |
| Correction value | ±4 | ±3 | ±1 | ±3 | ±2 | ±3 | ±5 |

In the aforementioned embodiment, the sampling time ranges are not overlapped. However, in other embodiment, the sampling time ranges may also be overlapped, which is not limited by the invention.

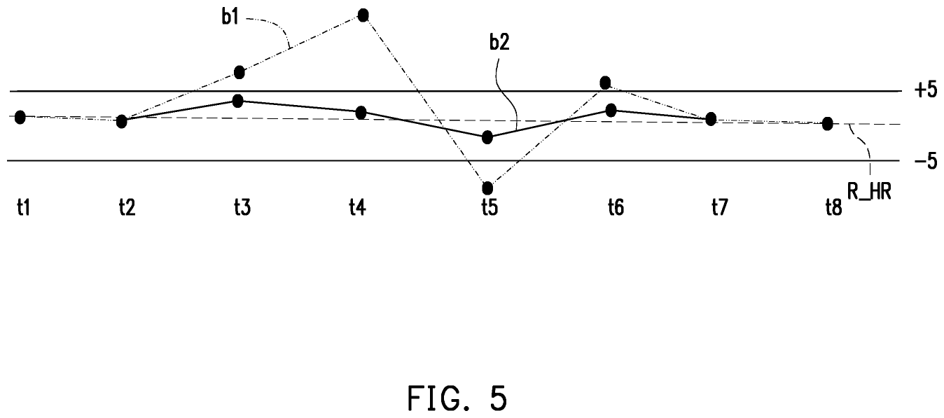

FIG. 5 is a diagram illustrating curves of output heart rates before and after correction according to an embodiment of the invention. In the embodiment, a sampling time range t1 to a sampling time range t8 of another embodiment are taken as an example for description. In FIG. 5, a curve b1 of uncorrected average heart rates and a curve b2 of corrected heart rates of the sampling time range t1 to the sampling time range t8 are illustrated. In the embodiment, the correction value ±5 is taken as an example for description. In the sampling time ranges t1, t2, t7, and t8, the average heart rates are directly taken as the output heart rates. Corrected heart rates of the output heart rates corresponding to the sampling time ranges t3 to t6 are obtained based on a reference heart rate R_HR and a correction value ±5. The average heart rates of the sampling time ranges t3, t4 and t6 are greater than the reference heart rate, so that the correction value is +5; and the average heart rate of the sampling time range t5 is smaller than the reference heart rate, so that the correction value is −5. Moreover, taking the sampling time ranges t3 and t4 as an example, a sampling credibility corresponding to the sampling time range t3 is higher than a sampling credibility corresponding to the sampling time range t4.

The higher the sampling credibility is, the lower a shaking degree of the user's body is, and the lower the sampling credibility is, the higher the shaking degree of the user's body is. When the sampling credibility is lower, if the difference between the average heart rate and the reference heart rate exceeds the variation range, it represents that a probability of being incredible is higher, so the correspondingly used correction value is smaller.

Further, the invention provides a computer readable medium, which includes a computer program product for executing the aforementioned heart rate correction method. The computer program product is basically composed of a plurality of code snippets (for example, an organization chart establishing code snippet, a table approving code snippet, a setting code snippet, and a deployment code snippet, etc), and these code snippets are loaded into an electronic device and executed by the same to accomplish various steps of the aforementioned heart rate correction method and various functions of the aforementioned heart rate correction system 100.

In summary, the invention provides the heart rate correction method and system to resolve the problem of heart rate accuracy reduction occurred when a static algorithm is adopted, when the user has a large sudden or temporary motion (an intensity of the acceleration detection signal is high), the previous heart rate value is used as a basis, and the current acceleration detection signal is referred to define the credibility of the instant heart rate. Then, an adjustment range of the heart rate is determined based on the credibility, but this method is also different from a method of direct subtraction of an ordinary dynamic algorithm, so that even if there is a motion in the static state, a stable heart rate value may still be output.

What is claimed is:

1. A heart rate correction method, comprising:
collecting a heartbeat measurement signal during a sampling time range to calculate a plurality of instant heart rates corresponding to a plurality of sampling intervals included in the sampling time range;
obtaining an acceleration detection signal during the sampling time range;
determining whether a credibility of each of the instant heart rates is credible or incredible based on the acceleration detection signal;
calculating an average heart rate of the instant heart rates of which the credibility is determined to be credible;
determining whether a difference between the average heart rate and a reference heart rate exceeds a variation range; and
obtaining a corrected heart rate based on a correction value and the reference heart rate when it is determined that the difference between the average heart rate and the reference heart rate exceeds the variation range, and taking the corrected heart rate as an output heart rate corresponding to the sampling time range.

2. The heart rate correction method as claimed in claim 1, wherein the step of determining whether the credibility of each of the instant heart rates is credible or incredible based on the acceleration detection signal comprises:
calculating an acceleration average of each of the sampling intervals;
comparing the acceleration average with a credibility threshold;
determining the credibility to be credible when the acceleration average is smaller than or equal to the credibility threshold; and
determining the credibility to be incredible when the acceleration average is greater than the credibility threshold.

3. The heart rate correction method as claimed in claim 1, further comprising:
storing the output heart rate in a temporary storage;
wherein before the step of determining whether the difference between the average heart rate and the reference heart rate exceeds the variation range, the heart rate correction method further comprises:
determining whether any of the output heart rate is stored in the temporary storage;
not to correct the average heart rate but directly taking the average heart rate as the output heart rate for storing in the temporary storage when it is determined that no output heart rate is stored in the temporary storage; and
calculating a heart rate average of all output heart rates included in the temporary storage when it is determined that any of the output heart rate is stored in the temporary storage, and taking the heart rate average as the reference heart rate to read the reference heart rate from the temporary storage to perform heart rate correction.

4. The heart rate correction method as claimed in claim 3, further comprising:
calculating a sampling credibility corresponding to the sampling time range based on a number of the instant heart rates of which the credibility is determined to be credible;
wherein when it is determined that no output heart rate is stored in the temporary storage, the heart rate correction method further comprises:
determining whether the sampling credibility is greater than a credibility threshold;
discarding storing the average heart rate in the temporary storage when it is determined that the sampling credibility is not greater than the credibility threshold; and
directly taking the average heart rate as the output hear rate for storing in the temporary storage when it is determined that the sampling credibility is greater than the credibility threshold.

5. The heart rate correction method as claimed in claim 4, wherein the sampling credibility is obtained based on a following equation:
Tr=N_true/N_sum;
wherein Tr represents the sampling credibility, N_true represents the number of the instant heart rates of which the credibility is determined to be credible, and N_sum represents a total number of the instant heart rates included in the sampling time range.

6. The heart rate correction method as claimed in claim 1, further comprising:
directly taking the average heart rate as the output heart rate corresponding to the sampling time range when it is determined that the difference between the average heart rate and the reference heart rate falls within the variation range.

7. The heart rate correction method as claimed in claim 1, further comprising:
calculating a sampling credibility corresponding to the sampling time range based on a number of the instant heart rates of which the credibility is determined to be credible;
wherein the step of obtaining the corrected heart rate based on the correction value and the reference heart rate further comprises:
obtaining the corresponding correction value from a correction table based on the sampling credibility, wherein the correction table records a plurality of credibility ranges and the respective corresponding correction value thereof.

8. A heart rate correction system, comprising:
a heart rate sensor;
an acceleration sensor; and
a processor, electrically coupled to the heart rate sensor and the acceleration sensor, wherein the processor is configured to:
collect a heartbeat measurement signal through the heart rate sensor during a sampling time range to calculate a plurality of instant heart rates corresponding to a plurality of sampling intervals included in the sampling time range;
obtain an acceleration detection signal through the acceleration sensor during the sampling time range;
determine whether a credibility of each of the instant heart rates is credible or incredible based on the acceleration detection signal;
calculate an average heart rate by taking out the instant heart rates corresponding to the sampling intervals of which the credibility is determined to be credible;
determine whether a difference between the average heart rate and a reference heart rate exceeds a variation range; and
obtain a corrected heart rate based on a correction value and the reference heart rate when it is determined that the difference between the average heart rate and the reference heart rate exceeds the variation range, and take the corrected heart rate as an output heart rate corresponding to the sampling time range.

9. The heart rate correction system as claimed in claim 8, wherein the processor is configured to:
calculate an acceleration average of each of the sampling intervals;
compare the acceleration average with a credibility threshold;
determine the credibility to be credible if the acceleration average is smaller than the credibility threshold; and
determine the credibility to be incredible if the acceleration average is greater than the credibility threshold.

10. The heart rate correction system as claimed in claim 8, wherein the processor is configured to:
store the output heart rate in a temporary storage;
wherein before determining whether the difference between the average heart rate and the reference heart rate exceeds the variation range, the processor is configured to:
determine whether any of the output heart rate is stored in the temporary storage;
not to correct the average heart rate but directly take the average heart rate as the output heart rate for storing in the temporary storage when it is determined that no output heart rate is stored in the temporary storage; and
calculate a heart rate average of all output heart rates included in the temporary storage when it is determined that any of the output heart rate is stored in the temporary storage, and take the heart rate average as the reference heart rate to read the reference heart rate from the temporary storage to perform heart rate correction.

11. The heart rate correction system as claimed in claim 10, wherein the processor is configured to:
calculate a sampling credibility corresponding to the sampling time range based on a number of the instant heart rates of which the credibility is determined to be credible;
wherein when it is determined that no output heart rate is stored in the temporary storage, the processor is configured to:
determine whether the sampling credibility is greater than a credibility threshold;
discard storing the average heart rate in the temporary storage when it is determined that the sampling credibility is not greater than the credibility threshold; and
directly take the average heart rate as the output hear rate for storing in the temporary storage when it is determined that the sampling credibility is greater than the credibility threshold.

12. The heart rate correction system as claimed in claim 11, wherein the sampling credibility is obtained based on a following equation:
Tr=N_true/N_sum;
wherein Tr represents the sampling credibility, N_true represents the number of the instant heart rates of which the credibility is determined to be credible, and N_sum represents a total number of the instant heart rates included in the sampling time range.

13. The heart rate correction system as claimed in claim 8, wherein the processor is configured to:
directly take the average heart rate as the output heart rate corresponding to the sampling time range when it is determined that the difference between the average heart rate and the reference heart rate falls within the variation range.

14. The heart rate correction system as claimed in claim 8, wherein the processor is configured to:
calculate a sampling credibility corresponding to the sampling time range based on a number of the instant heart rates of which the credibility is determined to be credible; and
obtain the corresponding correction value from a correction table based on the sampling credibility, wherein the correction table records a plurality of credibility ranges and the respective corresponding correction value thereof.

15. A heart rate correction system, comprising a processor electrically coupled to a heart rate sensor and an acceleration sensor, wherein the processor is configured to:
collect a heartbeat measurement signal through the heart rate sensor during a sampling time range to calculate a plurality of instant heart rates corresponding to a plurality of sampling intervals included in the sampling time range;
obtain an acceleration detection signal through the acceleration sensor during the sampling time range;
determine whether a credibility of each of the instant heart rates is credible or incredible based on the acceleration detection signal;
calculate an average heart rate by taking out the instant heart rates corresponding to the sampling interval of which the credibility is determined to be credible;
determine whether a difference between the average heart rate and a reference heart rate exceeds a variation range; and
obtain a corrected heart rate based on a correction value and the reference heart rate when it is determined that the difference between the average heart rate and the reference heart rate exceeds the variation range, and take the corrected heart rate as an output heart rate corresponding to the sampling time range.

16. The heart rate correction system as claimed in claim 15, wherein the processor is configured to:
calculate an acceleration average of each of the sampling intervals;
compare the acceleration average with a credibility threshold;
determine the credibility to be credible if the acceleration average is smaller than the credibility threshold; and
determine the credibility to be incredible if the acceleration average is greater than the credibility threshold.

17. The heart rate correction system as claimed in claim 15, wherein the processor is configured to:
store the output heart rate in a temporary storage;
wherein before determining whether the difference between the average heart rate and the reference heart rate exceeds the variation range, the processor is configured to:
determine whether any of the output heart rate is stored in the temporary storage;
not to correct the average heart rate but directly take the average heart rate as the output heart rate for storing in the temporary storage when it is determined that no output heart rate is stored in the temporary storage; and
calculate a heart rate average of all output heart rates included in the temporary storage when it is determined that any of the output heart rate is stored in the temporary storage, and take the heart rate average as the reference heart rate to read the reference heart rate from the temporary storage to perform heart rate correction.

18. The heart rate correction system as claimed in claim 15, wherein the processor is configured to:
calculate a sampling credibility corresponding to the sampling time range based on a number of the instant heart rates of which the credibility is determined to be credible;
wherein when it is determined that no output heart rate is stored in the temporary storage, the processor is configured to:
determine whether the sampling credibility is greater than a credibility threshold;
discard storing the average heart rate in the temporary storage when it is determined that the sampling credibility is not greater than the credibility threshold; and
directly take the average heart rate as the output hear rate for storing in the temporary storage when it is determined that the sampling credibility is greater than the credibility threshold.

19. The heart rate correction system as claimed in claim 15, wherein the processor is configured to:
calculate a sampling credibility corresponding to the sampling time range based on a number of the instant heart rates of which the credibility is determined to be credible; and
obtain the corresponding correction value from a correction table based on the sampling credibility, wherein the correction table records a plurality of credibility ranges and the respective corresponding correction value thereof.

20. A computer readable medium, storing a plurality of code snippets and loading the code snippets via an electronic device to execute following steps, comprising:
collecting a heartbeat measurement signal during a sampling time range to calculate a plurality of instant heart rates corresponding to a plurality of sampling intervals included in the sampling time range;
obtaining an acceleration detection signal during the sampling time range;
determining whether a credibility of each of the instant heart rates is credible or incredible based on the acceleration detection signal;
calculating an average heart rate by taking out the instant heart rates of which the credibility is determined to be credible;
determining whether a difference between the average heart rate and a reference heart rate exceeds a variation range; and
obtaining a corrected heart rate based on a correction value and the reference heart rate when it is determined that the difference between the average heart rate and the reference heart rate exceeds the variation range, and taking the corrected heart rate as an output heart rate corresponding to the sampling time range.
